(19) United States
(12) Patent Application Publication
(10) Pub. No.: US 2010/0121284 A1
(43) Pub. Date: May 13, 2010

(54) CANNULA FOR CUTANEOUS FILLERS

(76) Inventor: Doris Hexsel, Porto Alegre (BR)

Correspondence Address: LAMBERT & ASSOCIATES, P.L.L.C., 92 STATE STREET, BOSTON, MA, 02109-2004, US

(21) Appl. No.: 12/311,617

(22) PCT Filed: Oct. 5, 2007
(86) PCT No.: PCT/BR2007/000273

§ 371 Date: Jan. 4, 2010

(30) Foreign Application Priority Data
Oct. 5, 2006 (BR) MU8602090-0

(51) Int. Cl. A61M 25/00

(52) U.S. Cl. 604/264

(57) ABSTRACT

Cannula for cutaneous fillers comprising cannula with short stems, whose calibers vary from 16G to 24G, with a rounded, atraumatic tip containing an orifice on the distal extremity of vary shapes and sizes, the stem being straight, curved or angulated. The cannulae are provided with a detachable portion, which is composed of a plastic or metal sheath which is fixed to the extremities of ordinary syringes or to Luer Lock syringes which possess a mark indicating the position of the orifice.

### BACKGROUND OF THE INVENTION

The object of the present petition for patent for a utility model is comprised of a device to be used in introducing cutaneous fillers in patients in order to obtain aesthetic correction of wrinkles, furrows and tissue loss presented by said patients, and refers even more specifically to a short cannula of small caliber, with rounded ends and special orifices for use with cutaneous fillers. Techniques for application of cutaneous filling are minimally invasive surgical techniques used to apply the “fillers”. The fillers themselves are substances which are classified as exogenous or edogenous, biological or synthetic, and are used to augment volume in specific, localized areas, correcting wrinkles, furrows and volume which is lost either through trauma, leaving sequelas and scars, or through tissue loss typical of the aging of the face. It should be pointed out that fillers are especially used in the facial area, but may also be utilized in other body areas, being normally commercialized in gel forms of differing viscosities.

### THE STATE OF THE ART TECHNIQUE TODAY

Fillers are normally implanted using needles of different calibers. The needles permit implantation of these materials in the areas they are more frequently used with relative safety, but which offer risks when implanted in other parts of the body. There are innumerous cannulae described for different uses, including those used for filling with autologous fat. The smallest of these cannulae measure several centimeters in length and have calibers that make them inadequate for the implanting of most of the fillers that exist today and, above all, for those implants destined for the face.

Several documents for patents having as claims cannulae and other injecting devices for introduction of substances into the human body, do exist, such as the JP2005349179, which aims to promote a gentler removal of the cannula being used; the PI 9002870-8, which is a spatulated needle; the PI 9505238-0, which describes a barrier for the tip of the needle for a cannula; and the American document U.S. Pat. No. 6,706,017.

In regards to fillers, it should be pointed out that, in general, they are commercialized in small quantities (0.5 a 1 ml) and at an elevated cost, keeping in mind that they are designated for use in facial areas and are normally implanted in the dermis or subcutaneously using needles. When implants of large volumes are desired, or when thicker fillers are used, needles of a higher caliber are needed, and may damage veins and cause complications which vary from simple bruising to problems of a more serious nature, such as when the filler is accidentally deposited inside a vein.

Many of the collateral effects and complications connected with the use of fillers are directly linked to vascular lesions, or problems resulting from these lesions, be it for the fact that the needles have perforated veins, or due to accidental injection of these materials into the veins. A few examples follow.

The most common complication, and one of lesser gravity for the patient, is the simple perforation of a vein found in the area to be filled, resulting in a loss of blood to the dermis and subcutaneous are provoking hematomas, seromas and a type of residual pigmentation called haemosiderosis, which may linger from a few days to a few months respectively, finally disappearing spontaneously and leaving no sequelas.

A more serious complication can occur if a thicker type of filler is injected inside a vein, potentially causing embolism, whose clinical repercussions are diverse in both type and seriousness, depending on the location where the material has caused the obstruction of the vein. A thicker filler, or one that has been injected in a larger volume inside the vein, may cause damage to local circulation in varying degrees, ranging from reduction to complete loss of blood circulation in tissues thereby irrigated. In the case of total obstruction, necrosis can occur in the area irrigated by the vein involved. This filler injected inside a vein may be transported by blood circulation to other undesirable areas, provoking complications in other areas served by the vein under discussion and which may be equally severe. For example, one may cite the glabella area, the region between the eyebrows and a frequent target for fillers, where the venous drainage reports to the brain and the retina; a filler injected in a vein in the region may even cause blindness.

These complications may occur with any type of filler, even with autologous materials, such as fat, but are of greater severity when occurring with definitive fillers (non-absorbable) and those of thicker consistency.

There are cannulae used for filling with fat which may also be utilized for other types of fillers, but the thick caliber of the stems of these cannulae found on the market require larger incisions to permit penetration of the skin and may cause structural lesions along the trajectory traversed by the cannulae. Small scars, always undesirable on the face, where most filling takes place, may also result from the orifice wherein the cannula is introduced. These cannulae are also long, with a length varying in general from 5 to 20 or more centimeters, offering the additional problem of a greater waste of material, which remains retained between the syringe and the tip of the cannula. Here one points out that most of the implants are highly expensive and that the most recent are commercialized in small volumes (in general, 0.5, 1 or 2 ml), so that any waste, no matter how small, represents a reduction in cost/benefit for both doctors and patients.

Cannulas normally used for liposuction and fat injections are non-cutting instruments whose tips are usually rounded and, for this reason, are called “atraumatic”. They have long stems measuring from 5 to 20 centimeters or more in length and come with one or multiple round or oval orifices through which fat is aspirated and/or injected, being normally used in areas or situations in which the needles with their cutting tips can provoke lesions and/or collateral effects.

### NOVELTY AND USES OF THE PROPOSED MODEL

To resolve the problems above described, invention of special, delicate cannula of small caliber with rounded tips and orifices which permit the release of viscous materials having a gel-like consistency, is proposed, said cannulae herein referred to as “P cannulae”, with said cannulae being designated to implant fillers while aiming to minimize risk of vascular lesions and/or intravascular implant of fillers available on the market, preventing the above0cited complications and, in addition, allowing economizing of material which remains retained inside cannulae presently in use, with its subsequent waste.

The proposed cannulae are distinguished by their small size, short length, small caliber and rounded tips with versatile orifices allowing the implant of fillers free of trauma for veins and tissues subject to implants. The Cannulae in screen have been specially designed and will be used for dermis and subcutaneous implants so as to minimize risks of intravascular injection, responsible for the majority of the adverse reactions linked with the use of fillers, as well as minimizing waste of these expensive substances. Easily handled and delicate, said cannulae have no bevel or sharp angles capable of causing lesions to veins or tissues and will preferably be disposable. Their small calibers permit passage of most of the fillers on the market today, through a small incision in the skin, which may be effected with a needle, thereby avoiding scarring. The cannulae will be preferentially resterilizable or will be disposable, and will come individually packed and presterilized with ethylene oxide, remaining therefore sterile until the package is opened or shelf life expires, usually in three years,

The P cannulae possess a part capable of being attached to Luer Lock syringes (illustrated in attached drawings 1 to 5) and which may be made of metal, plastic or similar materials, like common needles, permitting that they may be used to substitute the needles normally used with the syringes proper for each type of filler. Alternately, they may suffer variations in form and may be attached directly to the syringes of the fillers, becoming a single unit, in which case, they will not be detachable.

### DESCRIPTION OF THE ANNEXED DRAWINGS

The annexed drawings illustrate the cannulae with the part that is attachable to syringes and the cannulae directly attached to the syringes, showing the different options for manufacture. In order that the present invention, object of this disclosure, be fully comprehended and put to use by any technician familiar with this technological sector, it will be explained clearly, concisely and sufficiently enough so as to permit its reproduction, based on the annexed drawings listed below:

FIGS. 1, 2 and 3 show the variations of the cannulae P in three sizes;

FIG. 4 shows an angled variation;

FIG. 5 shows a curved variation of the proposed invention;

FIG. 6 shows a cannula directly attached to the syringe.

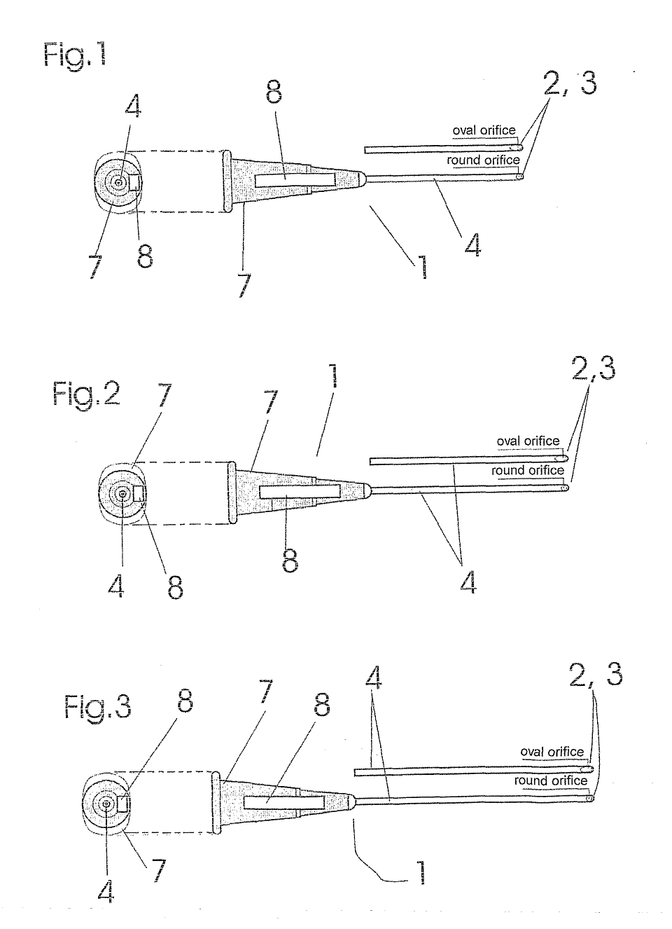

FIG. 1 shows a view from the top and a lateral view of a straight cannula P 2 cm long, in which the widest part, which attaches to the syringe, shows a mark that indicates the position of the orifice of the cannula P. The side view shows the same mark on the attachable part coincides with the two types of orifices (oval and round) and in addition, shows the possible variations in caliber of the cannula P, ranging from 24 G to 16 G.

FIG. 2 shows a view of the top and a lateral view of a straight cannula P 2.5 cm long, in which the widest part, that which attaches to the syringe, shows a mark indicating the position of the orifice of the cannula P. The lateral view shows that the same mark in the attachable part coincides with the two types of orifices (oval and round) and yet shows the possibilities for variations in the calibers of the cannula P, ranging from 24 G to 16 G.

FIG. 3 shows a view of the top and a lateral view of a straight cannula P 3 cm long, in which the widest part, that which attaches to the syringe, shows a mark indicating the position of the orifice of the cannula P. The lateral view shows that the same mark in the attachable part coincides with the two types of orifices (oval and round) and yet shows the possibilities for variations in the calibers of the cannula P, ranging from 24 G to 16 G.

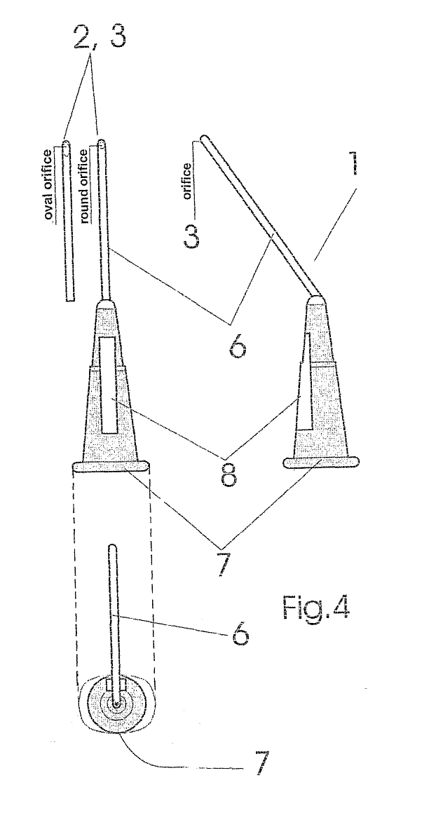

FIG. 4 show the angulated variation of the cannula P in which the above view shows the widest part that attaches to the syringe and in which the longest part is that of the cannula proper. The mark on the attachable part indicates the position of the orifice of the cannula P, as well as the direction of the angle. The lateral view shows that the same mark on the attachable part coincides with the two types of orifices (oval and round) and shows the possibilities of variations both in the calibers of the cannulae P, ranging from 24 G to 16 G, and also in the lengths from 2 to 3 cm, while the lateral inferior view shows in more detail the angulated variation of the cannula P, showing that the stem has an angle which can also vary, depending on its location and use.

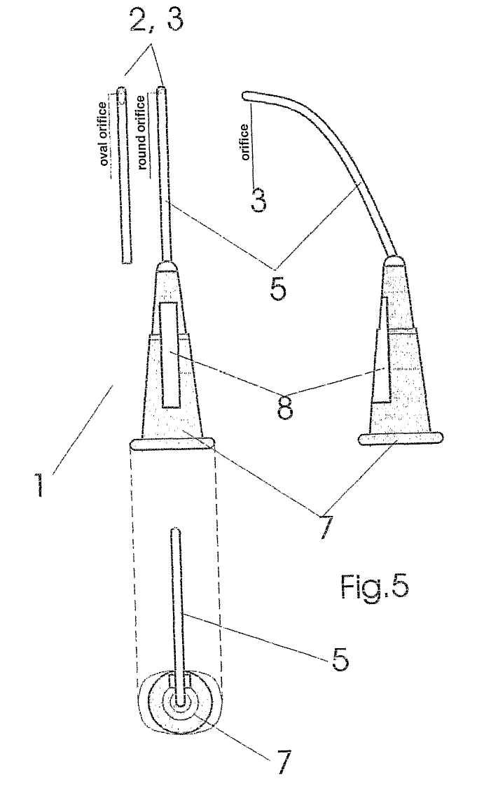

FIG. 5 shows the curved variation of the cannula P, in which the top view illustrates the widest part that attaches to the syringe and the longer part, which is that of the cannula proper. The mark on the attachable part indicates the position of the orifice of the cannula P, as well as the direction of the curve of the cannula P. The side view shows that the same mark on the attachable part coincides with the two types of orifices (oval and round) and shows the possibilities of variations in the calibers. The mark on the attachable part indicates the position of the orifice of the cannula P and also the direction of the cure of the cannula P. The side view shows that the same mark on the attachable part coincides with the two types of orifices (oval and round) and shows the possibilities of the variations in the calibers of the cannulae P, ranging from 24 G to 16 G, as well as the variations in the lengths, from 2 to 3 cm. The lower lateral view shows in more detail the angulated variation of the cannula P, showing that the stem has an angle, which can also vary, depending on locations and use.

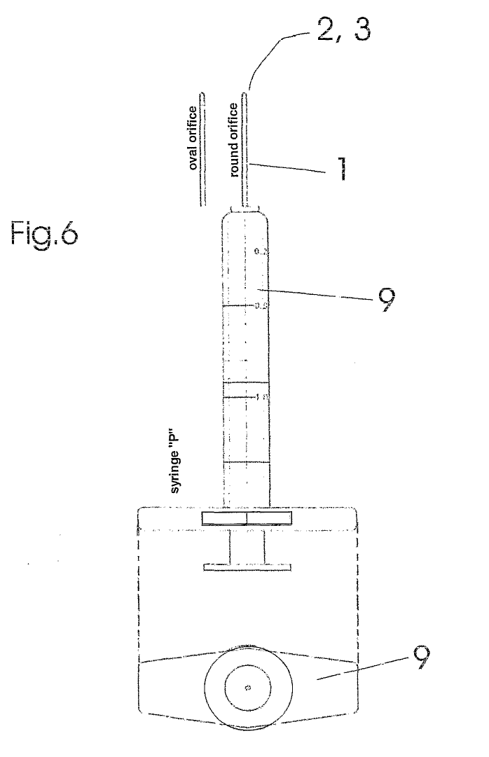

FIG. 6 shows the cannula P attached directly to the syringe, herewith called the “P syringe”, in which the attachable part has been suppressed so that the cannula P can be directly attached to the syringe. All variations shows in the figures described above, that is, length, caliber, size, as well as the sizes and shapes of the orifices and the shape of the stems can be directly attached in this variation.

### DETAILED DESCRIPTION OF SPECIFIC EMBODIMENTS

As can be understood by the annexed figures, the disposition of the cannula for cutaneous filling, object of this descriptive report, refers to a cannula (1) with a short stem which can measure from 2 to 3 cm in length, with calibers varying from 16 G to 24 G, with an atraumatic, rounded tip (2), containing an orifice in the distal extremity (3) of varying shapes and sizes (oval, round, square, rectangular, etc.), which allows passage of conventional fillers, such that the stem can be either straight (4), curved (5), or angulated (6), and with a part that can be attached to a syringe, and which indicates the position of the orifice of the cannula P.

The cannulae (1) have an attachable part (7) that is comprised of a plastic or metal connector that is affixed to the extremities of ordinary syringes or to Luer Lock syringes, and whose interior walls are smooth and straight, so that there are no corners permitting deposit and subsequent waste of filler. This attachable part (7) carries a mark (8) indicating the position of the orifice that allows the medical doctor to decide if the procedure will be done with the orifice turned down or up, obtaining more precision in the chosen course of action. Alternately, the attachable part (7) can be eventually suppressed, in case the stems of the cannulae (4 or 5 or 6) are affixed directly to the extremity of a syringe (9) and unable to be removed.

The cannulae (1) adopt lengths varying from 2 to 3 cm, in order to avoid waste of the materials or fillers that would otherwise be retained in the needle or cannula and therefore go unused; this length also allows access to the dermis and the subcutaneous dermis, or even deeper access, such as in applications near to the bone.

The stems can be straight (4) or curved (5), angulated (6) or not. The curved version (5), depicted in FIG. 5, in particular, facilitates better access to curved areas of the face, such as the back of the nose. The angulated stems inclined (6) permit oblique injections at certain anatomical areas, while the curved stems (5) allow implants in sinuous surfaces, such as the base of the nose and the malar regions.

The calibers of the cannulae (1) vary between 16 G, 18 G, 21 G and 24 G permitting that fillers be introduced through orifices so small that they will not leave scars; this variation of caliber permit the passage of the entire amount of almost all of the fillers commercialized around the world today whose viscosity is similar to that of a gel and are implanted through needles of these calibers or even smaller.

The tip (2) of the cannula is (1) rounded and substitutes the cutting bevel of the normal needle, while the orifice of the distal extremity (3) can have forms and shapes varying in accordance with the type of application planned. The sizes of the orifices (3) vary in accordance with that permitted by the caliber of the needles, allowing the fillers to be injected in varying quantities. The round orifice allows delivery of the material, permitting the implant in the form of a thin “thread”, similar to that of a needle, while the oval or rectangular orifice allows implant of the filler in a ribbon-like shape. This also allows control of the amount of material that can be implanted by these cannulae, which might be greater than that which can be obtained by the implant of fillers by bevels of needles of the same caliber, but with the advantages previously cited. The larger orifices, rectangular, allow the release of materials of lesser viscosity, which allows them to be compared to needles of higher caliber, in terms of the quantity of these materials that can be introduced via these orifices.

In the variance on the invention represented in FIG. 6, the cannula (10) is attached directly to the syringes. All cannula P models can be attached to the extremity of the syringe, that is the cannulae P of different sizes, stem formats, calibers and different sizes and formats of orifices can be attached to all models of syringes found on the market.

This description of the specific embodiments represents a new invention with a new functional improvement, with an inventive step and industrial application, all necessary prerequisites for the concession of the present patent request.

1. CANNULA FOR CUTANEOUS FILLERS, characterized by a cannula for cutaneous fillers (1) with a short, tubular conduit or stem, straight (4), curved (5) or angled (6), measuring from 2 to 3 cm in length with calibers ranging from 16 G to 24 G; with an atraumatic, rounded tip (2) containing an orfice or nozzle on the distal extremity (3) of varying sizes which may be oval, round, square or rectangular, presenting a connecting part (7) comprised of a connector with smooth, straight internal walls which holds the extremities of the syringes, which carry an external mark (8), indicating the coupling position of the orifice.

2. CANNULA FOR CUTANEOUS FILLERS, according to claim 1, and further characterized by alternatively the connecting segment (7) be eventually suppressed and the stems of the cannulas (4 or 5 or 6) directly fixed to the extremity of a syringe (9).
